(19) United States
(12) Patent Application Publication
(10) Pub. No.: US 2026/0085352 A1
(43) Pub. Date: Mar. 26, 2026

(54) PULSED RECHARGING FOR NANOPORE DEVICES

(71) Applicant: ILLUMINA, INC., San Diego, CA (US)

(72) Inventors: Christian Urban, San Diego, CA (US); Birch Simpson, San Diego, CA (US)

(21) Appl. No.: 19/315,251

(22) Filed: Aug. 29, 2025

Related U.S. Application Data
(60) Provisional application No. 63/699,460, filed on Sep. 26, 2024.

(51) Int. Cl. C12Q 1/6874

(52) U.S. Cl. CPC C12Q 1/6874

(57) ABSTRACT

Systems and methods for nanopore sequencing are disclosed. The systems and methods are related to using pulsed voltage waveforms to replenish depleted ions in nanopore unit cells.

### CROSS-REFERENCE TO RELATED APPLICATIONS

This application claims priority to U.S. Provisional Application No. 63/699,460, filed Sep. 26, 2024, the content of which is incorporated by reference in its entirety.

### BACKGROUND

### Field

The present technology generally relates to devices for determining the sequence of a biopolymer, such as a polynucleotide or polypeptide, and more specifically to nanopore sequencing devices.

### Description of the Related Art

Various polynucleotide sequencing techniques involve performing a large number of controlled reactions on support surfaces or within predefined reaction chambers. The controlled reactions may then be observed or detected, and subsequent analysis may help identify the properties of the polynucleotide involved in the reaction. Examples of such sequencing techniques include next-generation sequencing or massive parallel sequencing involving sequencing-by-ligation, sequencing-by-synthesis, reversible terminator chemistry, or pyrosequencing approaches.

Some polynucleotide sequencing techniques utilize a nanopore, which can provide a path for an ionic current. For example, as the polynucleotide traverses through the nanopore, it influences the ionic current through the nanopore. Each nucleotide, or series of nucleotides, that translocate relative to the nanopore yields a characteristic electrical signal. These characteristic electrical signals, as a result of the traversing polynucleotide, can be recorded to determine the sequence of the polynucleotide.

### SUMMARY

Provided herein are devices and method for sequencing biopolymers such as polynucleotides and polypeptide.

The devices and methods disclosed herein each have several aspects, no single one of which is solely responsible for their desirable attributes. Without limiting the scope of the claims, some prominent features will now be discussed briefly. Numerous other examples are also contemplated, including examples that have fewer, additional, and/or different components, steps, features, objects, benefits, and advantages. The components, aspects, and steps may also be arranged and ordered differently. After considering this discussion, and particularly after reading the section entitled “Detailed Description,” one will understand how the features of the devices and methods disclosed herein provide advantages over other known devices and methods.

It is to be understood that any features of the device and/or of the array disclosed herein may be combined together in any desirable manner and/or configuration. Further, it is to be understood that any features of the method of using the device may be combined together in any desirable manner. Moreover, it is to be understood that any combination of features of this method and/or of the device may be used together and/or may be combined with any of the examples disclosed herein. Still, further, it is to be understood that any feature or combination of features of any of the devices and/or of the arrays and/or of any of the methods may be combined together in any desirable manner and/or may be combined with any of the examples disclosed herein.

It should be appreciated that all combinations of the foregoing embodiments and additional embodiments discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A illustrates one embodiment of a nanopore sequencing system.

FIG. 1B illustrates a portion of one of the nanopore unit cells of the example nanopore sequencing system of FIG. 1A.

FIG. 2 illustrates a cross-sectional view of one embodiment of a nanopore sensor device.

FIG. 3 is illustrating an equivalent circuit diagram of the nanopore sensor device of FIG. 2.

FIG. 4 depicts the different modes of operation in a nanopore unit cell during an example course of sequencing.

FIGS. 5A-5C illustrate ion depletion and recharging during an exemplary applied AC voltage waveform.

FIGS. 6A-6C illustrate an exemplary applied voltage waveform used in open pore recharging.

FIG. 7 is a current-voltage plot of a MspA pore protein in 1M KCl buffer.

FIG. 8 shows an example pulsed voltage waveform.

FIG. 9A and FIG. 9B show a direct comparison between the step recharging scenario versus the pulsed recharging scenario.

FIG. 10A and FIG. 10B show a summary of experimental data testing the pulsed recharging scenario versus the step recharging scenario.

### DETAILED DESCRIPTION

All patents, applications, published applications, and other publications referred to herein are incorporated herein by reference to the referenced material and in their entirety. If a term or phrase is used herein in a way that is contrary to or otherwise inconsistent with a definition set forth in the patents, applications, published applications, and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, the term “electric connection” and the like refer to two spatial regions being connected together such that electrons, holes, ions or other charge carriers may flow between the two spatial regions.

If an electrolyte flows between two connected wells, ionic current may also flow between the connected wells. In some examples, two spatial regions may be in fluid/ionic/electric communication through one or more nanoscale openings or through one or more valves, restrictors, or other fluidic components that are to control or regulate a flow of fluid, ionic current through a system.

As used herein, the term “operably connected” refers to a configuration of elements, wherein an action or reaction of one element affects another element, but in a manner that preserves each element's functionality.

As used herein, the term “membrane” refers to a non-permeable or semi-permeable barrier or other sheet that separates two liquid/gel chambers (e.g., a cis well and a trans well) which can contain the same compositions or different compositions therein. Any membrane may be used in accordance with the present disclosure, as long as the membrane can include a transmembrane nanoscale opening and can maintain a potential difference across the membrane. The membrane may be a monolayer or a multilayer membrane. A multilayer membrane includes two or more layers. The membrane may be formed of materials of non-biological or biological origin. An example membrane that is made from non-biological materials is an amphiphilic polymer membrane.

An example membrane that is made from non-biological materials are block copolymer membranes. The term is a “block copolymer” is intended to refer to a polymer having at least a first portion or block that includes a first type of monomer, and at least a second portion or block that is coupled directly or indirectly to the first portion and includes a second, different type of monomer. Block copolymers include, but are not limited to, diblock copolymers and triblock copolymers. A “diblock copolymer” is intended to refer to a block copolymer that includes a first and second blocks coupled directly or indirectly to one another. The first block may be hydrophilic and the second block may be hydrophobic, in which case the diblock copolymer may be referred to as an “AB” copolymer where “A” refers to the hydrophilic block and “B” refers to the hydrophobic block. A “triblock copolymer” is intended to refer to a block copolymer that includes a first, second, and third blocks coupled directly or indirectly to one another. The first and third blocks may include, or may consist essentially of, the same type of monomer (repeating unit) as one another, and the second block may include a different type of monomer (repeating unit). In one example, the first block may be hydrophilic, the second block may be hydrophobic, and the third block may be hydrophilic and includes the same type of monomer as the first block, in which case the triblock copolymer may be referred to as an “ABA” copolymer where “A” refers to the hydrophilic blocks and “B” refers to the hydrophobic block. The block copolymers may be formed into a bilayer membrane in which the hydrophilic blocks are positioned on the outward of the bilayer membrane and in which the hydrophobic blocks are positioned inward of the bilayer membrane. For example, the membrane may be made of any diblock or triblock copolymers described in International Patent Application Publication No. WO2023/187104, the disclosure of which is incorporated herein by reference.

Example hydrophilic A blocks include, but are not limited to, a polymer selected from the group consisting of: N-vinyl pyrrolidone, polyacrylamide, zwitterionic polymer (Zwitt), hydrophilic polypeptide, poly(ethylene glycol) (PEG), carbon-oxygen-nitrogen containing polymers (CₓO_{y}N_{z}), polyacrylic acid, and combinations thereof. Example hydrophobic B blocks include, but are not limited to, poly (dimethylsiloxane) (PDMS), poly (isobutylene) (PiB), polybutadiene (PBd), polyisoprene, polymyrcene, polychloroprene, hydrogenated polydiene, polystyrene, fluorinated polyethylene, polypeptide, and combination thereof. Example block copolymers used to form a bilayer membrane include, but are not limited to, PDMS-ab-Zwitt, PiB-ab-Zwitt, PiB-ab-PEG, PiB-ab-(CₓO_{y}N_{z}), PDMS-ab-PEG, PDMS-ab-(CxOyNz), PiB-aba-Zwitt, PiB-aba-PEG, PiB-aba-(CₓO_{y}N_{z}), and PDMS-aba-PEG, PDMS-ab-(CxOyNz), and other suitable block copolymers,

An example membrane that is made from non-biological materials may be made from solid-state materials. The solid-state membrane can be a monolayer, such as a coating or film on a supporting substrate (i.e., a solid support), or a freestanding element. The solid-state membrane can also be a composite of multilayered materials in a sandwich configuration. Any material not of biological origin may be used, as long as the resulting membrane can include a transmembrane nanoscale opening and can maintain a potential difference across the membrane. The membranes may include organic materials, inorganic materials, or both. Examples of suitable solid-state materials include, for example, microelectronic materials, insulating materials (e.g., silicon nitride (Si₃N₄), aluminum oxide (Al₂O₃), hafnium oxide (HfO₂), tantalum pentoxide (Ta₂O₅), silicon oxide (SiO₂), etc.), some organic and inorganic polymers (e.g., polyamide, plastics, such as polytetrafluoroethylene (PTFE), or elastomers, such as two-component addition-cure silicone rubber), and glasses. In addition, the solid-state membrane can be made from a monolayer of graphene, which is an atomically thin sheet of carbon atoms densely packed into a two-dimensional honeycomb lattice, a multilayer of graphene, or one or more layers of graphene mixed with one or more layers of other solid-state materials. A graphene-containing solid-state membrane can include at least one graphene layer that is a graphene nanoribbon or graphene nanogap, which can be used as an electrical sensor to characterize the target polynucleotide. It is to be understood that the solid-state membrane can be made by any suitable method, for example, chemical vapor deposition (CVD). In an example, a graphene membrane can be prepared through either CVD or exfoliation from graphite.

A material that is of biological origin refers to material derived from or isolated from a biological environment such as an organism or cell, or a synthetically manufactured version of a biologically available structure (e.g., a biomimetic material).

An example membrane that is made from the material of biological origin includes a monolayer formed by a bolalipid. Another example membrane that is made from the material of biological origin includes a lipid bilayer. Suitable lipid bilayers include, for example, a membrane of a cell, a membrane of an organelle, a liposome, a planar lipid bilayer, and a supported lipid bilayer. A lipid bilayer can be formed, for example, from two opposing layers of phospholipids, which are arranged such that their hydrophobic tail groups face towards each other to form a hydrophobic interior, whereas the hydrophilic head groups of the lipids face outwards towards the aqueous environment on each side of the bilayer. Lipid bilayers also can be formed, for example, by a method in which a lipid monolayer is carried on an aqueous solution/air interface past either side of an aperture that is substantially perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has at least partially evaporated, the solution/air interfaces on either side of the aperture are physically moved up and down past the aperture until a bilayer is formed. Other suitable methods of bilayer formation include tip-dipping, painting bilayers, and patch-clamping of liposome bilayers. Any other methods for obtaining or generating lipid bilayers may also be used.

As used herein, the term “nanopore” is intended to mean a hollow structure discrete from, or defined in, and extending across the membrane. The nanopore permits ions, electric current, and/or fluids to cross from one side of the membrane to the other side of the membrane. For example, a membrane that inhibits the passage of ions or water-soluble molecules can include a nanopore structure that extends across the membrane to permit the passage (through a nanoscale opening extending through the nanopore structure) of the ions or water-soluble molecules from one side of the membrane to the other side of the membrane. The diameter of the nanoscale opening extending through the nanopore structure can vary along its length (i.e., from one side of the membrane to the other side of the membrane), but at any point is on the nanoscale (i.e., from about 1 nm to about 100 nm, or to less than 1000 nm). Examples of the nanopore include, for example, biological nanopores, solid-state nanopores, and biological and solid-state hybrid nanopores. In some embodiments, a nanopore refers to a pore having an opening with a diameter at its most narrow point of about 0.3 nm to about 2 nm. For example, a nanopore may be a solid-state nanopore, a graphene nanopore, an elastomer nanopore, or may be a naturally-occurring or recombinant protein that forms a tunnel upon insertion into a bilayer, thin film, membrane, or solid-state aperture, also referred to as a protein pore or protein nanopore herein (e.g., a transmembrane pore). If the protein inserts into the membrane, then the protein is a tunnel-forming protein.

As used herein, the term “diameter” is intended to mean a longest straight line inscribable in a cross-section of a nanoscale opening through a centroid of the cross-section of the nanoscale opening. It is to be understood that the nanoscale opening may or may not have a circular or substantially circular cross-section. Further, the cross-section may be regularly or irregularly shaped.

As used herein, the term “biological nanopore” is intended to mean a nanopore whose structure portion is made from materials of biological origin. Biological origin refers to a material derived from or isolated from a biological environment such as an organism or cell, or a synthetically manufactured version of a biologically available structure. Biological nanopores include, for example, polypeptide nanopores and polynucleotide nanopores.

As used herein, the term “polypeptide nanopore” is intended to mean a protein/polypeptide that extends across the membrane, and permits ions, electric current, polymers such as DNA or peptides, or other molecules of appropriate dimension and charge, and/or fluids to flow therethrough from one side of the membrane to the other side of the membrane. A polypeptide nanopore can be a monomer, a homopolymer, or a heteropolymer. Structures of polypeptide nanopores include, for example, an a-helix bundle nanopore and a β-barrel nanopore. Example polypeptide nanopores include a-hemolysin, Mycobacterium smegmatis porin A (MspA), gramicidin A, maltoporin, OmpF, OmpC, PhoE, Tsx, F-pilus, acrolysin, CsgG, etc. The protein α-hemolysin is found naturally in cell membranes, where it acts as a pore for ions or molecules to be transported in and out of cells. Mycobacterium smegmatis porin A (MspA) is a membrane porin produced by Mycobacteria, which allows hydrophilic molecules to enter the bacterium. MspA forms a tightly interconnected octamer and transmembrane beta-barrel that resembles a goblet and contains a central pore.

In various embodiments, any suitable pore protein may be used for the nanopore, including wild-type pore proteins. In some embodiments, the pore protein may be mutated (e.g., to enhance polynucleotide translocation). In some embodiments, the pore protein may not be functionalized. For example, the pore protein may be wild-type MspA porins, mutant MspA porins, wild-type MspA paralog porins, wild-type MspA homolog porins, mutant MspA paralog porins, mutant MspA homolog porins, single-chain Msp porins, or any pore protein described in U.S. Pat. No. 8,673,550 or any polypeptide nanopore described in International Patent Application Publication No. WO2022/211998, the disclosure of each of which is incorporated herein by reference.

A polypeptide nanopore can be synthetic. A synthetic polypeptide nanopore includes a protein-like amino acid sequence that does not occur in nature. The protein-like amino acid sequence may include some of the amino acids that are known to exist but do not form the basis of proteins (i.e., non-proteinogenic amino acids). The protein-like amino acid sequence may be artificially synthesized rather than expressed in an organism and then purified/isolated.

As used herein, the term “polynucleotide nanopore” is intended to include a polynucleotide that extends across the membrane, and permits ions, ionic current, and/or fluids to flow from one side of the membrane to the other side of the membrane. A polynucleotide pore can include, for example, a polynucleotide origami (e.g., nanoscale folding of DNA to create the nanopore).

As used herein, the term “solid-state nanopore” is intended to mean a nanopore whose structure portion is defined by a solid-state membrane and includes materials of non-biological origin (i.e., not of biological origin). A solid-state nanopore can be formed of an inorganic or organic material. Solid-state nanopores include, for example, silicon nitride nanopores, silicon dioxide nanopores, solid-state nanopores in polymeric membranes (e.g., polyimide), and graphene nanopores.

The nanopores disclosed herein may be hybrid nanopores. A “hybrid nanopore” refers to a nanopore including materials of both biological and non-biological origins. An example of a hybrid nanopore includes a polypeptide-solid-state hybrid nanopore and a polynucleotide-solid-state nanopore.

The application of the potential difference across a nanopore may force the translocation of a nucleic acid or a polynucleotide through or relative to the nanopore. One or more signals are generated that correspond to the translocation of the nucleotide through or relative to the nanopore. Accordingly, as a target polynucleotide, or as a mononucleotide or a probe derived from the target polynucleotide or mononucleotide, transits through or relative to the nanopore, the current across the membrane changes due to base-dependent (or probe dependent) blockage of the nanopore constriction, for example. The signal from that change in current can be measured using any of a variety of methods. Each signal is unique to the species of nucleotide(s) (or probe) in the nanopore, such that the resultant signal can be used to determine a characteristic of the polynucleotide. For example, the identity of one or more species of nucleotide(s) (or probe) that produces a characteristic signal can be determined. The polynucleotide may or may not completely translocate through the nanopore and out into the trans well.

As used herein, the term “nanopore sequencer” or “nanopore sensor device” refers to any of the devices disclosed herein that can be used for nanopore sequencing. In the examples disclosed herein, during nanopore sequencing, the nanopore is immersed in an electrolyte and a potential difference is applied across the membrane. In an example, the potential difference is an electric potential difference or an electrochemical potential difference. An electrical potential difference can be imposed across the membrane via a voltage source that injects or administers current to at least one of the ions of the electrolyte contained in the cis well or one or more of the trans wells. An electrochemical potential difference can be established by a difference in ionic composition of the cis and trans wells in combination with an electrical potential. The different ionic composition can be, for example, different ions in each well or different concentrations of the same ions in each well. Apparatuses and methods include sequencing polynucleotides and sequencing polypeptides and include providing genomics analysis and proteomics analysis.

As used herein, a “peptide” refers to two or more amino acids joined together by an amide bond (that is, a “peptide bond”). Peptides comprise up to or include 50 amino acids. Peptides may be linear or cyclic. Peptides may be α, β, γ, δ, or higher, or mixed. Peptides may comprise any mixture of amino acids as defined herein, such as comprising any combination of D, L, α, β, γ, δ, or higher amino acids.

As used herein, a “protein” refers to an amino acid sequence having 51 or more amino acids.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen, containing heterocyclic base, can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides are natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-cthano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing. The term “nucleic acid” may be used interchangeably with “polynucleotide” to refer to a deoxyribonucleotide or ribonucleotide polymer in either single- or double-stranded form and, unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

For example, a template polynucleotide chain may be any sample that is to be sequenced, and may be composed of DNA, RNA, or analogs thereof (e.g., peptide nucleic acids). The source of the template (or target) polynucleotide chain can be genomic DNA, messenger RNA, or other nucleic acids from native sources. In some cases, the template polynucleotide chain that is derived from such sources can be amplified prior to use. Any of a variety of known amplification techniques can be used including, but not limited to, polymerase chain reaction (PCR), rolling circle amplification (RCA), multiple displacement amplification (MDA), or random primer amplification (RPA). It is to be understood that amplification of the template polynucleotide chain prior to use is optional. As such, the template polynucleotide chain will not be amplified prior to use in some examples. Template/target polynucleotide chains can optionally be derived from synthetic libraries. Synthetic nucleic acids can have native DNA or RNA compositions or can be analogs thereof.

In some examples, template polynucleotide chains can be obtained as fragments of one or more larger nucleic acids. Fragmentation can be carried out using any of a variety of techniques known in the art including, for example, nebulization, sonication, chemical cleavage, enzymatic cleavage, or physical shearing. Fragmentation may also result from use of a particular amplification technique that produces amplicons by copying only a portion of a larger nucleic acid chain. For example, PCR amplification produces fragments having a size defined by the length of the nucleotide sequence on the original template that is between the locations where flanking primers hybridize during amplification. The length of the template polynucleotide chain may be in terms of the number of nucleotides or in terms of a metric length (e.g., nanometers).

A population of template/target polynucleotide chains, or amplicons thereof, can have an average strand length that is desired or appropriate for a particular sequencing device. For example, the average strand length can be less than about 100,000 nucleotides, about 50,000 nucleotides, about 10,000 nucleotides, about 5,000 nucleotides, about 1,000 nucleotides, about 500 nucleotides, about 100 nucleotides, or about 50 nucleotides. Alternatively or additionally, the average strand length can be greater than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. Alternatively or additionally, the average strand length can be greater than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. Alternatively or additionally, the average strand length can be greater than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The average strand length for a population of target polynucleotide chains, or amplicons thereof, can be in a range between a maximum and minimum value set forth above.

In some cases, a population of template/target polynucleotide chains can be produced under conditions or otherwise configured to have a maximum length for its members. For example, the maximum length for the members can be less than about 100,000 nucleotides, about 50,000 nucleotides, about 10,000 nucleotides, about 5,000 nucleotides, about 1,000 nucleotides, about 500 nucleotides, about 100 nucleotides or about 50 nucleotides. For example, the maximum length for the members can be less than about 100,000 kilo nucleotides, about 50,000 kilo nucleotides, about 10,000 kilo nucleotides, about 5,000 kilo nucleotides, about 1,000 kilo nucleotides, about 500 kilo nucleotides, about 100 kilo nucleotides or about 50 kilo nucleotides. For example, the maximum length for the members can be less than about 100,000 mega nucleotides, about 50,000 mega nucleotides, about 10,000 mega nucleotides, about 5,000 mega nucleotides, about 1,000 mega nucleotides, about 500 mega nucleotides, about 100 mega nucleotides or about 50 mega nucleotides. Alternatively or additionally, a population of template polynucleotide chains, or amplicons thereof, can be produced under conditions or otherwise configured to have a minimum length for its members. For example, the minimum length for the members can be more than about 10 nucleotides, about 50 nucleotides, about 100 nucleotides, about 500 nucleotides, about 1,000 nucleotides, about 5,000 nucleotides, about 10,000 nucleotides, about 50,000 nucleotides, or about 100,000 nucleotides. For example, the minimum length for the members can be more than about 10 kilo nucleotides, about 50 kilo nucleotides, about 100 kilo nucleotides, about 500 kilo nucleotides, about 1,000 kilo nucleotides, about 5,000 kilo nucleotides, about 10,000 kilo nucleotides, about 50,000 kilo nucleotides, or about 100,000 kilo nucleotides. For example, the minimum length for the members can be more than about 10 mega nucleotides, about 50 mega nucleotides, about 100 mega nucleotides, about 500 mega nucleotides, about 1,000 mega nucleotides, about 5,000 mega nucleotides, about 10,000 mega nucleotides, about 50,000 mega nucleotides, or about 100,000 mega nucleotides. The maximum and minimum strand length for template polynucleotide chains in a population can be in a range between a maximum and minimum value set forth above.

As used herein, the term “signal” is intended to mean an indicator that represents information. Signals include, for example, an electrical signal and an optical signal. The term “electrical signal” refers to an indicator of an electrical quality that represents information. The indicator can be, for example, current, voltage, tunneling, resistance, potential, conductance, inductance, impedance, or a transverse electrical effect (and any time-derivatives or transients of theses). An “electronic current” or “electric current” refers to a flow of electric charge. In an example, an electrical signal may represent an ionic current passing through a nanopore, and the ionic current may flow when an electric potential difference is applied across the nanopore.

The term “substrate” refers to a rigid, solid support that is insoluble in aqueous liquid and is incapable of passing a liquid absent an aperture, port, or other liquid conduit. In the examples disclosed herein, the substrate may have wells or chambers defined therein. Examples of suitable substrates include wafers, glass and modified or functionalized glass, plastics (including acrylics, polystyrene, and copolymers of styrene and other materials, polypropylene, polyethylene, polybutylene, polyurethanes, polytetrafluoroethylene (PTFE) (such as TEFLON® from Chemours), cyclic olefins/cyclo-olefin polymers (COP) (such as ZEONOR® from Zeon), polyimides, polymethyleneoxide, etc.), nylon, ceramics, silica or silica-based materials, silicon and modified silicon, carbon, metals, inorganic glasses, and optical fiber bundles.

The terms top, bottom, lower, upper, on, etc., are used herein to describe the device/nanopore sequencer and/or the various components of the device. It is to be understood that these directional terms are not meant to imply a specific orientation but are used to designate relative orientation between components. The use of directional terms should not be interpreted to limit the examples disclosed herein to any specific orientation(s). As used herein, the terms “upper,” “lower,” “vertical,” “horizontal” and the like are meant to indicate relative orientation.

As used herein, by “translocation,” it is meant that an analyte (e.g., a polynucleotide, such as DNA) moves relative the nanopore opening, but the analyte does not need to move through the nanopore and exit other side of the nanopore opening. For example, the analyte may enter any side of the nanopore, such as the cis side and/or trans side) and may optionally exit the other side of the nanopore opening. For example, the analyte may be translocated multiples times relative to the nanopore in one or more directions, such as cis-to-trans direction, trans-to-cis direction, or both. It is contemplated that any embodiment herein comprising translocation may refer to non-electrophoretic translocation or electrophoretic translocation, unless specifically noted.

As used herein, the terms “well,” “cavity,” and “chamber” are used synonymously and refer to a discrete feature defined in the device that can contain a fluid (e.g., liquid, gel, gas). A “cis well” is a chamber that contains or is partially defined by a cis electrode, and is also fluidically connected to a trans well through a respective nanopore. Examples of an array of the present device may comprise one or more cis wells where an individual cis well is a common chamber for a group of trans wells. Each “trans well” is a single chamber that contains or is partially defined by its own trans electrode and is also fluidically connected to a cis well. Each trans well is electrically isolated from the other trans well. In some examples, each trans well is connected to a respective stimulus source and to a respective amplifier (e.g., Axopatch 200B amplifiers) to amplify electrical signals passing through respective nanopores associated with each of the trans wells. In other examples, the trans wells are connected to a single stimulus source which individually addresses the trans wells via multiplexing. Further, it is to be understood that the cross-section of a well taken parallel to a surface of a substrate, at least partially defining the well, can be curved, square, polygonal, hyperbolic, conical, angular, etc.

The aspects and examples set forth herein and recited in the claims can be understood in view of the above definitions.

### Overview of Nanopore Sequencing

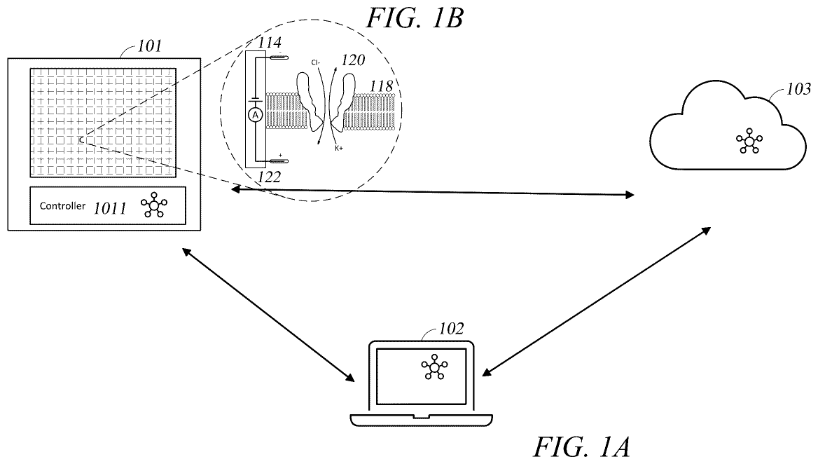

FIG. 1A illustrates an example nanopore sequencing system which may be used to implement some embodiments disclosed herein. The example nanopore sequencing system may include a nanopore sequencer 101, which may include a controller 1011 and an array of nanopore unit cells. The controller 1011 may be configured to control the sequencing operations in the array of nanopore unit cells. The example nanopore sequencing system may further include a computer 102 that is operably connected with the nanopore sequencer 101. The example nanopore sequencing system may further include a data storage and computing resource 103, such as a network or cloud, which may be operably connected with the nanopore sequencer 101 and the computer 102.

In some examples, the control functionalities of controller 1011 can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor or group of processors for performing the methods described herein may be of various types, including programmable devices (e.g., CPLDs and FPGAs) and non-programmable devices such as gate array ASICs or general-purpose microprocessors.

A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory. In some examples, a hardware platform for providing a computational environment may be used. The hardware platform may comprise a processor (e.g., CPU) and a memory such as random-access memory (RAM). In some embodiments, graphics processing units (GPUs) can be used. In some embodiments, hardware platforms for performing computational methods as described herein comprise one or more computer systems with one or more processors. In some embodiments, smaller computers are clustered together to yield a supercomputer network. The hardware platform may be specially constructed for the required purposes, or it may be a general-purpose computer (or a group of computers) selectively activated or reconfigured by a computer program and/or data structure stored in the computer. In some embodiments, a group of processors performs some or all of the described functionalities collaboratively (e.g., via a network or cloud computing) and/or in parallel.

Elements of the methods or processes described herein can be embodied in a software module executed by a processor. A software module can reside in RAM memory, flash memory, ROM memory, EPROM memory, EEPROM memory, registers, hard disk, a removable disk, a CD-ROM, or any other form of computer-readable storage medium. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions. Certain embodiments can implement at least one processor and memory to perform one or more aspects and examples, as disclosed herein. Embodiments described herein are not limited by the type or quantity of these processors and memory.

FIG. 1B illustrates an example of a portion of one of the nanopore unit cells. The nanopore unit cell may include a membrane 118. The membrane 118 can be formed from any suitable natural and/or synthetic material. The membrane 118 may also be formed of a non-permeable or semi-permeable material. In an example, the membrane 118 includes a block copolymer structure or a bilipid layer. The nanopore unit cell may further include a nanopore 120, which may be any of the biological nanopores, solid-state nanopores, hybrid nanopores, and synthetic nanopores. In an example, the nanopore 120 may be a hollow defined by, for example: a polynucleotide structure, a polypeptide structure, or a solid-state structure, e.g., a carbon nanotube, which is disposed in the membrane 118. In a further example, the membrane 118 may be a synthetic membrane (e.g., a solid-state membrane, one example of which is silicon nitride), and the nanopore 120 is in a hollow extending through the membrane 118. In some embodiments, a biological nanopore may be formed of peptides or polynucleotides and deposited in a block copolymer membrane, e.g., a synthetic polymeric membrane. In some embodiments, a solid-state nanopore may be formed as a nanoscale opening in a membrane (e.g., silicon-based, graphene, or polymer membrane).

The membrane 118 separates the nanopore unit cell into a cis compartment/well and a trans compartment/well. A target polynucleotide can translocate from the cis well, relative to the nanopore 120, to the trans well. A cis electrode 114 is associated with the cis compartment, and a trans electrode 122 is associated with the trans compartment. The electrodes may be used to apply a voltage across the nanopore, thus driving ionic current flows through the nanopore 120 and exerting an electric force on the target polynucleotide. In some examples, the electrodes are faradaic electrodes. In some examples, the electrodes are non-faradaic electrodes. A current detector may be used to measure the ionic current through the nanopore and the detected signal may be transmitted to the sequencer controller 1011.

When a bias is applied across the nanopore 120, a polynucleotide may translocate relative to the nanopore 120. In some embodiments, the polypeptide may translocate from the cis well, relative to the nanopore 120, toward the trans well, or vice versa. When a nucleotide or a sequence combination of nucleotides of the polynucleotide is in or near the nanopore, it may result in a unique ionic current blockade at the nanopore 120 and, therefore, a unique “nanopore resistance” depending on the identity of the nucleotide or the sequence combination of nucleotides. By measuring the ionic current or the “nanopore resistance”, the nucleotide or the sequence combination of nucleotides at or near the nanopore can be identified. In other words, the polynucleotide translocating relative to the nanopore may modulate the electrical properties of the nanopore such that the nucleobase sequence of the polynucleotide can be identified. For example, the current through the nanopore or the electrical resistance at the nanopore may be a function of the identity of the nucleobase of the polynucleotide at or near the nanopore. Furthermore, for the same polynucleotide, the “nanopore resistance” may be different when the polynucleotide is moving through the nanopore in different directions.

In some embodiments, the target polynucleotide may undergo base pairing during sequencing. In some embodiments, the nucleotides utilized for the base pairing may include a unique tag or label. The tag/label may also result in a unique ionic current blockade at the nanopore 120, and thus can be used for sequencing the target polynucleotide. In some embodiments, a portion of the polynucleotide may translocate through the nanopore from the cis well toward the trans well, but the polynucleotide as a whole does not exit the nanopore into the trans well. In other embodiments, the polynucleotide may translocate through the nanopore from cis well into the trans well.

In some embodiments, sequencing of a target polynucleotide may involve nanopore sensing of (1) a single-stranded portion of the target polynucleotide; (2) a nucleic acid duplex of a portion of the target poly nucleotide; (3) a label or tag that can be tethered or untethered to the target polynucleotide; (4) chemical modifications thereof, or (5) any combination thereof.

In some embodiments, multiple nanopore unit cells may be arranged in an array, and each nanopore unit cell may be individually accessed by a logic circuit. In some embodiments, a nanopore array may be present in a nanopore sensor device.

Although embodiments herein describe determining a signal level by determining the ionic current through the nanopore, embodiments also include alone or in combination determining the signal level by measuring other electrical characteristics of the nanopore unit cell. For example, in other embodiments, a signal level is determined by the voltage potential at a specified area or component of the nanopore cell. For example, in other embodiments, a signal level is determined by the conductivity or resistance of the nanopore's associated membrane. For example, in other embodiments, a signal level is determined by the electrical impedance at a specified area or component of the nanopore cell.

### Nanopore Sensor Device

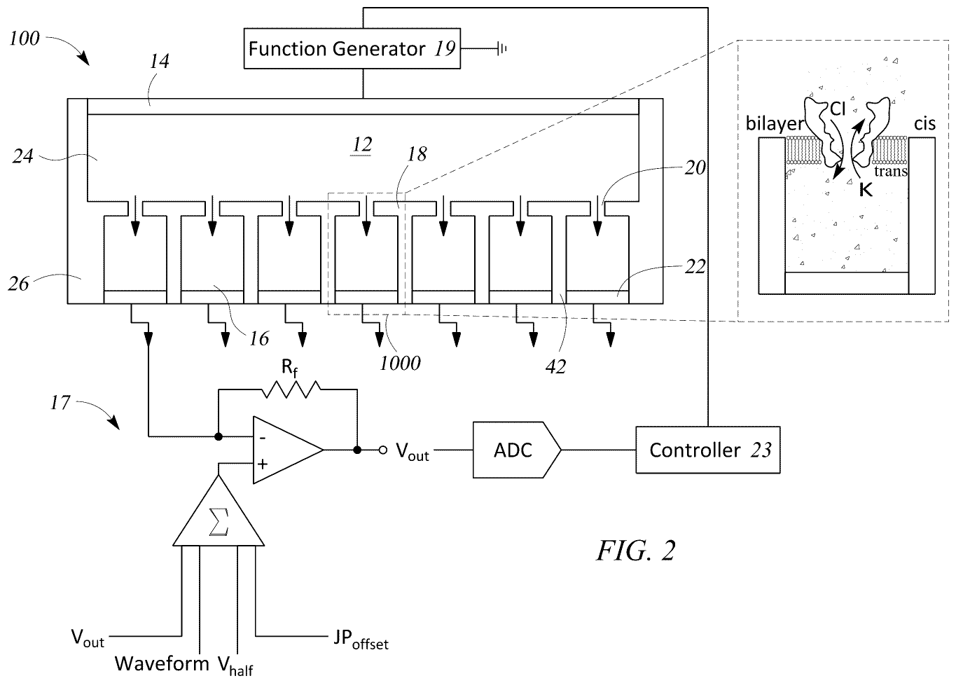

A nanopore sensor device may have a plurality of nanopore unit cells that forms a sensor array. FIG. 2 depicts an example of a nanopore sensor array 100 that contains a plurality of nanopore unit cells 1000. The exemplified nanopore array 100 include a cis well 12, a cis electrode 14, and a plurality of trans wells 16 separated from the cis well 12 by a polymer/lipid/solid-state/hybrid membrane 18. The membrane 18 contain a plurality of nanopores 20, each associated with a trans well 16 and an individually addressable trans electrode 22. The nanopore(s) 20 may be positioned in, and extend through, the membrane 18 to establish the fluidic connection between the cis well 12 and the trans well(s) 16. The trans wells 16 and the cis well 12 may be filled with an electrolyte solution 24. The trans wells 16 are each associated a trans electrode 22 and are individually addressable. The cis electrode 14 is implemented on the top of the cis well 12. In various embodiments, the width of the cis electrode 14 may be smaller than, larger than or equal to the size of the total or combined widths of the trans wells 16. Thus, a bias voltage is applied between the cis electrode 14 and individual trans electrode 22, which can cause flowing the ions in a direction based on the polarity of the applied bias voltage. In some embodiments, a positive ionic current is defined by positively charged ions flowing from the trans side of the nanopore to the cis side of the nanopore. For example, if a positive bias voltage is applied (e.g., trans electrode 22 has a positive polarity compared to the cis electrode 14), the negative charged anions (e.g., A− and polynucleotide molecule) in the cis well 12 may move toward the nanopore 20 and the trans well 16, thus generating an ionic current flowing from the trans well 16 to the cis well 12. If a negative bias voltage is applied (e.g., trans electrode 22 has a negative polarity compared to the cis electrode 14), the negative charged anions (e.g., A− and polynucleotide molecule) in the trans well 16 moves toward the nanopore 20 and the cis well 12, thus generating an ionic current flowing from the cis well 12 to the trans well 16.

The cis electrode 14 that is used depends, at least in part, upon the electrolyte species in the electrolyte solution. In some examples, the cis electrode 14 may be an active faradaic electrode that takes part in the chemical reaction with an electrochemically active electrolyte species and can be oxidized or reduced in the half-cell reaction. Examples of active electrodes include silver (Ag), copper (Cu), zinc (Zn), lead (Pb), intercalation electrodes (e.g. Prussian blue), redox polymers (e.g. PEDOT), etc. In other examples, the cis electrode 14 may be an inactive (or inert, or polarizable) electrode that transfers electrons rather than exchanges ions with the electrolyte solution 24. A polarizable electrode may also induce capacitive charging of ions at the electrode-electrolyte interface without directly exchanging ions with the electrolyte solution. Examples of inactive electrodes include platinum (Pt), carbon (C) (e.g., graphite, diamond, etc.), gold (Au), rhodium (Rh), etc. For example, in the nanopore sensor utilizing an electrolyte solution 24 with an electrically active anion (e.g., chloride, Cl⁻), the cis electrode 14 may be a silver/silver chloride (Ag/AgCl) electrode.

The trans electrode 22 that is used depends, at least in part, upon the electrolyte species in the electrolyte solution. The trans electrode 22 may be an active electrode that takes part in the chemical reaction with an electrochemically active electrolyte species, and can be oxidized or reduced in the half-cell reaction. Any of the examples of the active electrodes set forth herein for the cis electrode 14 may be used as the trans electrode 22. In other examples, the trans electrode 22 may be an inactive (or inert) electrode that transfers electrons rather than exchanges ions with the electrolyte solution 24. Any of the examples set forth herein for the cis electrode 14 may be used as the trans electrode 22. In an example, in the nanopore sensor utilizing an electrolyte solution 24 with an electrically active anion (e.g., chloride, Cl⁻), the trans electrode 22 may be a silver/silver chloride (Ag/AgCl) electrode.

Many different layouts of the trans wells 16 may be envisaged, including regular, repeating, and non-regular patterns. In an example, the trans wells 16 are disposed in a hexagonal grid for close packing and improved density. Other layouts may include, for example, rectilinear (i.e., rectangular) layouts, triangular layouts, and so forth. As examples, the layout or pattern can be an x-y format of trans wells 16 that are in rows and columns. In some other examples, the layout or pattern can be a repeating arrangement of trans wells 16 and/or interstitial regions 42. In still other examples, the layout or pattern can be a random arrangement of trans wells 16 and/or interstitial regions 42. The pattern may include spots, posts, stripes, swirls, lines, triangles, rectangles, circles, arcs, checks, plaids, diagonals, arrows, squares, and/or cross-hatches.

The layout may be characterized with respect to the density of the trans wells 16 (i.e., number of trans wells 16 in a defined area of the substrate 26). For example, the trans wells 16 may be present at a density ranging from about 10 wells per mm² to about 1,000,000 wells per mm². The density may be tuned to different densities, including, for example, a density of at least about 10 per mm², about 5,000 per mm², about 10,000 per mm², about 0.1 million per mm², or more. Alternatively or additionally, the density may be tuned to be no more than about 1,000,000 wells per mm², about 0.1 million per mm², about 10,000 per mm², about 5,000 per mm², or less. It is to be further understood that the density of the trans wells 16 in the support 26 can be between one of the lower values and one of the upper values selected from the ranges above.

The layout may also or alternatively be characterized in terms of the average pitch, i.e., the spacing from the center of a nanopore 20 to the center of an adjacent nanopore 20 (center-to-center spacing). The pattern can be regular such that the coefficient of variation around the average pitch is small, or the pattern can be non-regular in which case the coefficient of variation can be relatively large. In an example, the average pitch may range from about 100 nm to about 500 μm. The average pitch can be, for example, at least about 100 nm, about 5 μm, about 10 μm, about 100 μm, or more. Alternatively or additionally, the average pitch can be, for example, at most about 500 μm, about 100 μm, about 50 μm, about 10 μm, about 5 μm, or less. The average pitch for an example array including a particular pattern of nanopores 20 can be between one of the lower values and one of the upper values selected from the ranges above. In an example, the array has an average pitch (center-to-center spacing) of about 10 μm.

The trans wells 16 may be micro wells (having at least one dimension on the micron scale, e.g., about 1 μm up to, but not including, 1000 μm), such as from about 1 μm to about 750 μm, such as from about 5 μm to about 500 μm, such as from about 10 μm to about 250 μm, such as from about 15 μm to about 100 μm. Each trans well 16 may be characterized by its aspect ratio (e.g., width or diameter divided by depth or height, respectively). In an example, the aspect ratio of each trans well 16 may range from about 1:1 to about 1:5.

Each trans well 16 has an opening (e.g., that faces the cis well 12) that is large enough to accommodate at least a portion of the membrane 18 and the nanopore 20 that is associated therewith. For example, an end of the nanopore 20 may extend through the membrane 18 and into the opening of the trans well 16.

The cis well 12 and the trans wells 16 may be fabricated using a variety of techniques, including, for example, photolithography, nanoimprint lithography, stamping techniques, embossing techniques, molding techniques, microetching techniques, etc.

The membrane 18 may be any of the non-permeable or semi-permeable materials described herein. The membrane 18 is positioned between the cis well 12 and the trans wells 16, and thus provides a barrier between the wells 12, 16. The membrane may be positioned on the interstitial region 42 of the substrate 26.

The nanopore(s) 20 may be any of the biological nanopores, solid state nanopores, and hybrid nanopores described herein. As mentioned herein, each nanopore 20 fluidically connects a respective one of the trans wells 16 to the cis well 12. As such, the ratio of nanopores 20 to trans wells 16 is 1:1.

The nanopore 20 has two open ends and a hollow core or hole that connects the two open ends. The walls of the hollow core or hole are an inner surface of the nanopore 20. When inserted into the membrane 18, one of the open ends of the nanopore 20 faces the cis well 12 and the other of the open ends of the nanopore 20 faces the trans well 16. The hollow core of the nanopore 20 enables the fluidic connection between the wells 12, 16. The diameter of the hollow core may range from about 1 nm up to 1 μm, and may vary along the length of the nanopore 20. In some examples, the open end that faces the cis well 12 may be larger than the open end that faces the trans well 16. In other examples, the open end that faces the cis well 12 may be smaller than the open end that faces the trans well 16.

The nanopore(s) 20 may be inserted into the membrane 18, or the membrane 18 may be formed around the nanopore(s) 20. In an example, the nanopore 18 in its monomeric form or oligomeric form (e.g., an octamer, nonamer, etc.) may insert into a formed bilayer (one example of the membrane 18). For example, the nanopore may insert into a bilayer through electroporation, pipette pump cycle, and/or detergent assisted pore insertion. For example, an oligomeric nanopore 20 may assemble into a nanopore and insert into the bilayer. In another example, the nanopore 20 may be added to a well of a bilayer at a desirable concentration where it will insert into the bilayer. In yet a further example, the nanopore 20 may be inserted into to a solid support (e.g., silicon, silicon oxide, quartz, indium tin oxide, gold, polymer, etc.).

An anchoring molecule, which may be part of the nanopore 20 or may be attached to the nanopore 20, may attach the nanopore 20 to the solid support. The attachment via the anchoring molecule may be such that a single nanopore 20 is immobilized (e.g., between two chambers/wells). A bilayer may then be formed around the nanopore 20. The nanopore sensor device 100 includes an electrolyte solution 24 in the cis well 12 and the trans wells 16.

The electrolyte may be any electrolyte that is capable of dissociating into cations and anions. For example, the salt may include any suitable combination of cations (such as, but not limited to, H⁺, Li⁺, Na⁺, K⁺, NH₄⁺, Ag⁺, Ca²⁺⋅ Ba²⁺, and/or Mg²⁺) with any suitable combination of anions (such as, but not limited to, OH⁻, Cl⁻, Br⁻, I⁻, NO₃⁻, ClO₄⁻, F⁻, SO₄²⁻, and/or CO₃²⁻.). In one example, the electrolyte can be for an Ag/AgCl redox system. In another example, the electrolyte may also be one or more salts of a redox mediator system, such as ferrocyanide/ferricyanide redox couple in an electrolyte buffer in which ferrocyanide ions (e.g., Fe(CN)₆⁴⁻) are oxidized to ferricyanide ions (e.g., Fe(CN)₆³⁻) and ferricyanide ions (Fe(CN)₆³⁻) are reduced to ferrocyanide ions (Fe(CN)₆⁻). In this case, the mediator system may be combined with a secondary electrolyte species (e.g., KCl).

The nanopore sensor device 100 also includes electronics to individually and/or collectively address each of the trans electrodes 22. As mentioned herein, each of the trans electrodes 22 is associated with a respective trans well 16 and a respective nanopore 20. The electronics include at least a stimulus source and a controller. As shown in the embodiment of FIG. 2, a function generator 19 is applied to a common cis electrode 14 for a plurality of trans wells 16. As shown in the embodiment of FIG. 2, each of the trans electrodes comprises electronics 17 to individually address each of the trans electrodes 22 by applying a waveform and by individually measuring a signal from the trans electrode. A controller 23 is coupled to the function generator 19 and the electronics 17 to control current flow through an individual nanopore 20 by addressing an individual trans electrodes 22. The controller is coupled to the stimulus source, and the controller is configured to individually/selectively address one of the plurality of trans electrodes 22 (using the stimulus source) to cause an ionic current to flow through the nanopore 20 connected to the addressed trans electrode 22. The electronics may also include amplifier(s) to amplify electrical signals passing through respective nanopores 20 associated with trans electrodes 22 that are addressed.

In some embodiments, the stimulus source applies a voltage bias between the cis well 12 and at least one of the plurality of trans wells 16 (using the electrodes 14, 22), and thus across the nanopore 20 and the membrane 18. The voltage bias that is applied may be a positive polarity to the trans electrode 22 to attract negative charge compounds (such as negatively charge nucleotides, negatively charged labels/tags) in the cis well 12 towards the nanopore 20 and/or a negative polarity to the trans electrode 22 to repel negatively charged compounds (such as negatively charge nucleotides, negatively charged labels/tags) in the cis well 12 away from the nanopore 20. In some embodiments, the voltage bias ranges from about 10 V to about 10 V, from about −5 V to about 5 V, from about −2 V to about 2 V, from about 1 V to about 1 V, from about −200 mV to about 200 mV, −100 mV to about 100 mV between the cis electrode 14 and the trans electrodes 22 during a sensing or sequencing cycle. Any voltage bias within the given range may be applied during a sensing or sequencing cycle. For example, when measuring nucleobase identities (sequencing phase), the voltage bias may range from about −200 mV to about 200 mV; during non-measurement, such as ejection, capturing or recharging phases (which will be explained in more details below), the voltage bias may range from about 0 mV to about 500 mV, or from about −1000 mV to about 1000 mV in various phases. In certain examples, the nanopore sensor device 100 operates in a bipolar mode (e.g., alternating current), providing a negative bias and a positive bias to the trans electrode 22. In certain examples, the nanopore sensor device 100 operates in a unipolar mode (e.g., direct current), providing a positive bias to the trans electrode 22. In some instances, the voltage can be reduced, or the polarity reversed, to facilitate appropriate function.

In some embodiments, for manufacturing efficiency and cost saving purposes, it is desirable to have an external (i.e., off the chip) cis electrode as part of a microfluidics setup while having sensing electrodes/trans electrodes addressing each nanopore embedded in the chip. The cis electrode can be places several centimeters from the chip surface. A voltage can be applied between the cis electrode and the individual trans electrodes, and thus, an ionic current can pass through each nanopore in the array.

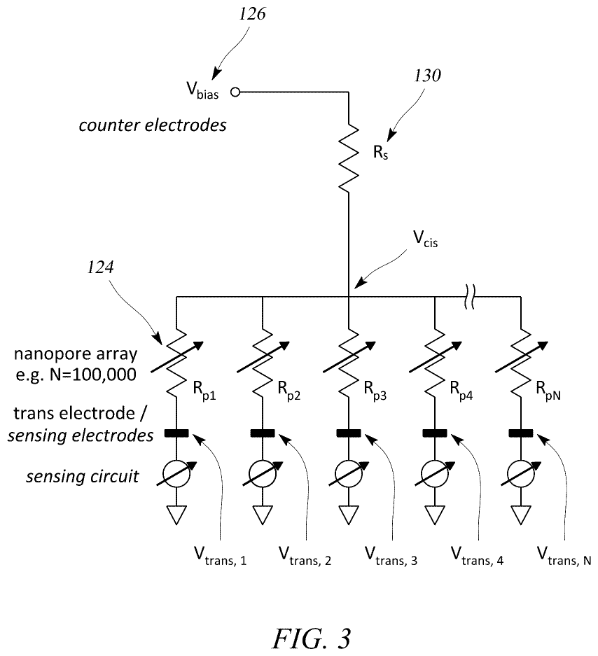

FIG. 3 illustrates an equivalent circuit diagram of the nanopore device 100 as depicted in FIG. 2. Each “nanopore resistance” Rₚᵢ 124 represents the resistance of a single nanopore unit cell, where i=1, 2, 3, . . . , N, and depends on the parameters of the unit cell and the states of the associated membrane, nanopore and polynucleotide in or near the nanopore. Different polynucleotide sequences can cause specific changes in the pore resistance (and thus observed pore current) as the polynucleotide translocate relative to the nanopore. In a sensor array, the collection of all nanopore unit cells can be viewed as an array of parallel resistors with a total resistance Rₚₜₒₜ, which can be calculated according to 1/Rₚₜₒₜ=1/Rₚ₁+1/Rₚ₂+ . . . +1/R_{pN}. Thus the total resistance Rₚₜₒₜ in a large array can be orders of magnitude smaller than that of single or few unit cell. For example, an array of 100,000 nanopore unit cells where each individual nanopore unit cell features a resistance of 1 GOhm would have a total resistance of only 10 kOhm. For large enough arrays of nanopore unit cells, the total resistance of all nanopore unit cells can therefore become comparable to the resistance of the electrolyte connection leading to the cis electrode. When a voltage Vbias 126 is applied to the cis electrode 14, the voltage drop across the buffer resistance Rₛ 130 can be calculated by Vₛ=Iₛ*Rₛ, where Iₛ is the current flowing through Rₛ (and equals to the sum of currents through each nanopore unit cells). As a result, the voltage at the cis end of the nanopores V_{cis} is therefore

$${V}_{cis} = {V}_{bias} - {V}_{s} .$$

As illustrated by FIG. 3, while all the nanopore unit cells share the same V_{cis}, the voltage applied to each individual trans electrode, V_{trans,i}, where i=1, 2, 3, . . . , N, are individually controlled (by an associated active circuitry in the ASIC layer). Therefore the voltage difference across each individual nanopore unit cell, V_{cis}−V_{trans,i}, can be individually controlled. This allows for independent actuation of the nanopore unit cells and the motion (e.g., direction and/or speed) of polynucleotides in nanopores. Therefore, the nanopore sequencing device shown in FIG. 2 can achieve asynchronous sequencing, i.e., sequencing at different unit cells may occur at different times.

In some instances, a sensor's bias voltage may be changed independently for a particular stage of the sequencing process. For example, individual sensors can be set to a number of different voltages to enable ejection of a used (i.e., completed sequencing) or stalled polynucleotide from a nanopore, replenishing of electrochemical components inside a cis/trans well, or capturing of a fresh polynucleotide in the nanopore. Depending on the bias state of a trans electrode the current going through the respective pore can become larger, smaller, and even opposite in sign to the typical current observed in the sequencing mode. In addition, it may be possible to turn off a nanopore sensor during a sequencing run if no nanopore is inserted into the membrane, if the membrane supporting the nanopore breaks, or if multiple pores are present in a membrane for an individual trans well. Different number of nanopores may be present in the array between unit cells since loading the individual channels/wells with nanopores is related to the fluid concentration of nanopores that is subject to process variations, which may lead to pore insertion in fewer than all of the desired individual channels/wells.

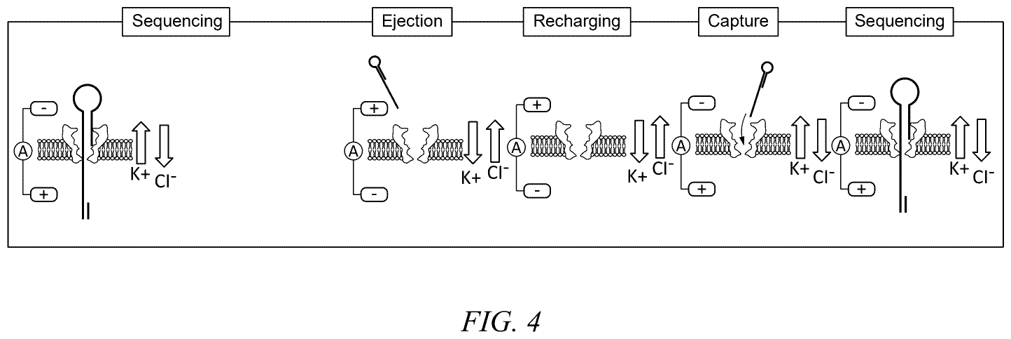

In some embodiments, the nanopore device correlates the measured electrical characteristics with the sequencing process. In these embodiments, the current flow direction can correspond to the stages of the sequencing process. For example, as shown in FIG. 4, when a positive bias is applied at the trans electrode relative to the cis electrode, anions can flow from the cis well to the trans well, which creates a “forward current.” A polynucleotide, being negatively charged, is also attracted to the positively biased trans electrode and moves from the cis well towards the trans well. This causes the polynucleotide to translocate relative to the nanopore towards the trans well. In another example, when the bias is reversed and a negative bias is applied at the trans electrode relative to the cis electrode, anions can flow from the trans well into the cis well. Similarly, the negatively charged polynucleotide is repelled by the negatively biased trans electrode and results in translocation of the polynucleotide relative to the nanopore away from the trans wells. This direction can generally be referred to as a “reverse current.”

In another example, during recharging stage, when the nanopore may be an open nanopore without a captured polynucleotide, the ionic concentrations in the cis and the trans wells may be rebalanced by negatively biasing the trans electrode to provide a reverse current moving anions from the trans well into the cis well. In some cases, during recharging stage the nanopore may have an analyte (e.g., polynucleotide) in it. In another example, during a capturing stage of the sequencing process of a polynucleotide introduced to a cis well, a positive bias is applied at the trans electrode to provide a forward current and to capture the polynucleotide by the nanopore of the trans well.

In some nanopore unit cells, no polynucleotide is captured by a nanopore which is inserted in a membrane; this situation is termed an “open pore” state and is associated with either a forward or a reverse current, depending on the polarity of the difference between Vₜᵣₐₙₛ and V_{cis}. In some nanopore unit cells, no nanopore is inserted in the membrane and thus no current can flow through the unit cell. If the magnitude of (Vₜᵣₐₙₛ and V_{cis}) increases beyond a threshold value, the membrane may break (or rupture or burst). In nanopore unit cells where membrane is broken or not painted, and thus also without pore protein, current flows in either the forward or the reverse direction, depending on the polarity of the difference between Vₜᵣₐₙₛ and V_{cis}. FIG. 4 is a schematic illustration of certain embodiments of sequencing using double-stranded and/or single stranded portions of DNA. Embodiments of present apparatus and methods includes (1) a single-stranded portion of the target polynucleotide; (2) a nucleic acid duplex of a portion of the target poly nucleotide; (3) a label or tag that can be tethered or untethered to the target polynucleotide; (4) chemical modifications thereof, or (5) any combination thereof. FIG. 4 illustrates certain embodiments wherein ejection occurs from the cis well with a reverse current. In other embodiments, ejection occurs from the trans well with a forward current. FIG. 4 illustrates certain embodiments wherein capture occurs from the cis well with a forward current. In other embodiments, capture occurs from the trans well with a reverse current. FIG. 4 illustrates the flow of anions during certain embodiments of sequencing, ejection, recharging, and capture in which the anions comprise chlorine (Cl⁻) anions and the cations comprise potassium (K⁺) ions). In other embodiments, the anions may be any suitable anions.

In some embodiments, changing the state for each nanopore unit cell can be performed by implementing a circuitry configured to control the state. The state, for example, can include sequencing, ejecting, recharging, and capturing. For example, during the sequencing and/or capturing states, the circuitry may generate positive bias voltage meaning that the voltage that enables the potential at trans wells is higher than the cis well. In another example, during the ejecting and/or recharging states, the circuitry may generate a negative bias to enable the cis well to have a higher potential than the trans well. The magnitude of the potential difference in each state can also be varied, such that the recharging state can have a higher magnitude than the sequencing state. In certain embodiments a controller, such as controller 1011 of FIG. 1A, of the instrument calculates the direction and the magnitude of voltage to apply to each wells/electrodes. In some embodiments, the controller may be a FPGA. Waveforms during sequencing can be DC, stepped, pulsed, AC, sinusoidal, or combinations thereof.

As explained above, during various stages of faradaic nanopore sequencing, predominantly positive currents pass through each nanopore unit cell of the nanopore sequencing device. Positive current is defined in connection with a positive Vₜᵣₐₙₛ−V_{cis}, i.e., the trans side has a higher electrical potential compared to the cis side. Under such an applied voltage difference, for example, the arrow in the nanopore unit cell 1000 shown in FIG. 2 indicates that a negatively charged polynucleotide would be translocating from the cis well to the trans well. In the meanwhile, anions (e.g., chloride) would be moving from the cis well to the trans well, and cations would be moving from the trans well to the cis well. Under such circumstances, the nanopore sequencing process may suffer from short-term electrolyte depletion and/or long-term electrode degradation.

As one of the consequences of the prolonged net positive bias applied to the trans electrodes, electrolyte may be depleted due to different ionic species being transported across the nanopore at different rates, which can cause a shift in electrolyte composition in the nanopore sensor's trans well. While this positive bias is necessary for capturing, reading, and advancing DNA constructs in the nanopore, it also causes an imbalance in the supply and consumption of ions at the trans wells. Over time, the concentration of active ions in the trans well is depleted. Electrolyte depletion could affect the pore conductivity due to asymmetric salt concentrations as well as the junction potential at the sensing electrode. In addition, electrolyte depletion may increase the resistance of the electrolyte in the trans well. Therefore, depletion of electrolyte ions in the trans wells may limit the runtime/lifetime of the nanopore sensor device by causing instabilities, such as a decay in current or deformation of the membrane that surrounds the nanopore (e.g., 18 in FIG. 2).

In some cases, if a constant DC bias voltage is used, the current would be decreased as a function of time due to electrolyte depletion. To compensate for the current decay, the applied voltage may need to be adjusted to increase over time to sustain adequate current levels for sequencing. However, increasing the applied voltage and thus the input power may be undesirable.

Nanopore sequencing platforms typically involve an array of individual trans wells each equipped with a unique trans electrode. In this array, many DNA strands can be sequenced in parallel at each well. The data throughput therefore depends on the density of trans wells packed into the footprint of the flow cell. Accordingly, it is beneficial to minimize the size of the trans well to maximize the density of the sequencing pixels. However, shrinking the trans well volume has been shown to accelerate electrolyte depletion, as the number of usable electrolyte ions is smaller in smaller wells.

### Recharging in Nanopore Device

Both short-term electrolyte depletion and long-term electrode degradation can be minimized by balancing the positive charges with negative ones, e.g., by flowing a negative current (reversing the current direction) through each nanopore unit cell.

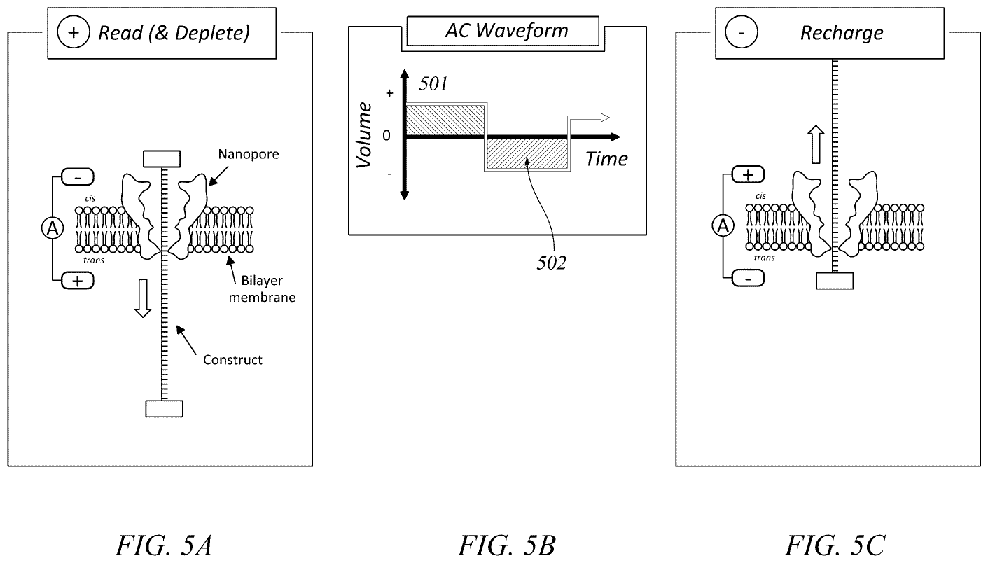

For example, one approach to mitigate electrolyte depletion is to apply an alternating current (AC) waveform in which an intermittent negative bias “recharges” the trans well. Examples of AC waveforms include sinusoidal waveform, a triangular waveform, a square waveform, a stepping waveform, or any combination thereof that comprises at least one instance of positive bias and at least one instance of a negative bias. The recharging step(s) in the AC waveform replenishes the ions in the trans well and reverses the depletion process. The utility of such an AC waveform is shown schematically in FIGS. 5A-5C. FIG. 5B shows a bipolar voltage waveform including a positive portion 501 and a negative portion 502. The positive portion 501 is applied in the case of FIG. 5A, while the negative portion 502 is applied in the case of FIG. 5C. As shown in FIG. 5A, the positive pulses 501 in the waveform which is primarily used for sequencing/reading, pulls the construct strand down into the nanopore, but also causes depletion of trans well ions. On the other hand, in FIG. 5C the negative portion 502 of the bipolar waveform, which pushes the construct upward out of the pore and into the cis well and can even be used to eject the strand, can be used for recharging the trans well. By adjusting how long the negative portion 502 of the voltage waveform is applied, for example, one can adjust the current and the ionic charge passing through the membrane during this stage to balance out the effect of charge depletion.

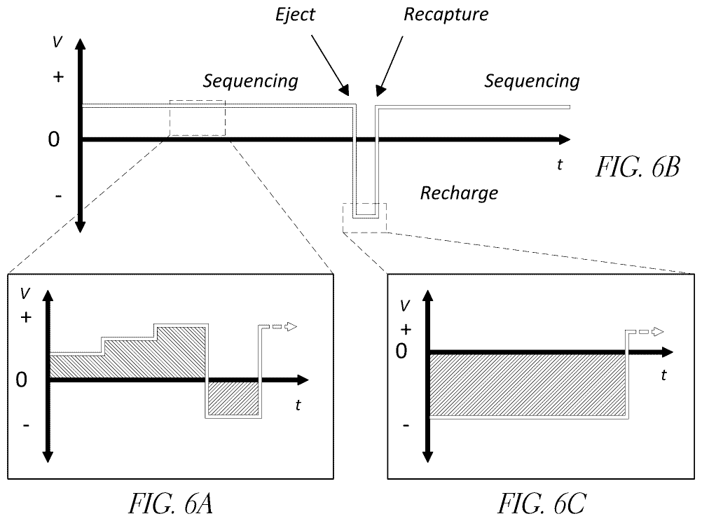

The trans well may also be recharged when the pore lacks any construct or template, i.e., in its “open” configuration. The key advantage of this approach is that the recharging process can happen much more quickly owing to the higher conductance of the open pore. Therefore, the overall sequencing rate will not be affected as drastically. A schematic showing open pore periodic recharging is presented in FIGS. 6A-6C. The sequencing process is performed with a waveform that is chiefly dictated by the biochemistry conditions without any restrictions/modifications made to fully recharge the system in real time, as shown in FIG. 6A. Then, the construct/template is ejected as indicated in FIG. 6B, and a waveform is applied to the open pore that induces an overall net negative charge passed to recharge the system, as shown in FIG. 6C. For example, a DC bias may be applied to recharge the system (e.g., −50 mV for 5 min), but any DC waveform, pulsed waveform, stepped waveform of a series of one or more steps, sweep waveform, or combinations thereof, or AC waveform (e.g. sinusoidal, triangular, square wave) may be used to recharge as long as the net charge passed during the recharging step is opposite that which is passed during sequencing. In the example case illustrated in FIG. 6B, the net charge passed during recharging is negative, while the net charge passed during sequencing is positive. After the recharging process is complete, a second construct or template is re-captured, and the process is repeated, as indicated in FIG. 6B.

For periodic recharging to be effective, the recharging process may occur before electrolyte depletion has any significant impact on the signal fidelity and base-calling results. Ideally, a single template/construct could be sequenced just until depletion starts to impact the read levels, at which point the template would be ejected and the trans well would be recharged. Therefore, the extent to which a given template may be sequenced continuously is ultimately still somewhat limited by depletion. So, while open pore recharging would extend the overall lifetime of the nanopore system itself, the duration of continuous sequencing on a single template, and therefore the maximum read length, are still constrained.

In some embodiments, open pore and blocked pore recharging can be combined to extend the maximum read length. The active charge passed during sequencing can be monitored so that when the system is nearing depletion, the trans well can be recharged by applying a negative voltage while a template is still in the pore. This would allow longer templates to be read, albeit at a slightly slower rate than without recharging. In some cases, by implementing this process, the overall sequencing process can be performed for at least seven 3× rounds of 10 min extensions (3.5 total sequencing time) on a 10 μm CD well. The runtime could potentially be extended indefinitely provided that other failure modes not relating to depletion (e.g., template unlocking, incomplete template ejection, stalling) are suppressed. Recharging may also be accomplished by modifying the underlying sequencing waveform (FIG. 6A) such that the positive and negative portions of the waveform are adequately balanced based on predictions of positive and negative currents that have passed during the respective portions, such that recharging occurs real-time within the sequencing process, and no periodic states designated exclusively to recharging are required.

Instead of performing recharge in a controlled voltage mode, where a defined voltage waveform is applied to induce estimated currents, the recharging process may also be performed in a controlled current (galvanostatic) mode. In a controlled current mode, a defined current waveform is applied for a duration of time. An example galvanostatic waveform may be a DC current waveform, but it also may be a stepped current waveform comprising one or more current steps, a pulsed current waveform, a sinusoidal current waveform, or a combination thereof. In this embodiment, the real voltage applied adjusts to sustain the pre-defined recharging current waveform. The waveform is applied for a duration of time such that the amount of charge passed during recharging is sufficient to rebalance the ion concentration in the trans well. The magnitude of the charge passed during the controlled current recharge must be opposite that passed during sequencing. For example, if net charge passed during sequencing is positive, then the net charge passed during recharging must be negative. In cases where the ion transference is equal under positive and negative bias, the duration and magnitude of the galvanostatic recharge waveform is adjusted to satisfy the condition of Area (A)=Area (B) in the current diagram, i.e., the consumed charge equals the replenished charge. In cases where the ion transference under positive and negative biases is unequal, satisfactory recharging conditions may be achieved where Area (A)≠Area (B). In these cases, depletion can be mitigated under conditions where Area (A)≠Area (B), but instead when the ratio of Area (A)/Area (B) is a number not equal to unity. For example, Arca (A)/Arca (B) may range from about 0.01 to about 1, or from about 1 to about 100. In these scenarios, the magnitude and duration of the controlled current recharging waveform may be chosen to satisfy such conditions.

### Pulsed Recharging

An additional technical challenge in the nanopore sequencing process is that the pore protein (e.g., MspA) may show gating behavior, such as voltage-dependent gating behavior. Negative currents that are more negative than a certain threshold can cause the nanopore to “gate” (e.g., opening/closing), which is due to conformational/structural change of the pore protein or charge rearrangement on the pore protein. As a result, nanopore conductivity may drop when a large negative voltage is applied across the nanopore, such as during recharging. Gating is usually associated with a dramatic increase in nanopore resistance and therefore creates a limit on the current that can be passed in the negative direction, which is determined at least in part by the magnitude of the bias voltage threshold. The bias voltage threshold is the bias voltage above which gating is not observed (i.e., gating is not observed when the bias voltage is more positive than the bias voltage threshold, typically having a negative value).

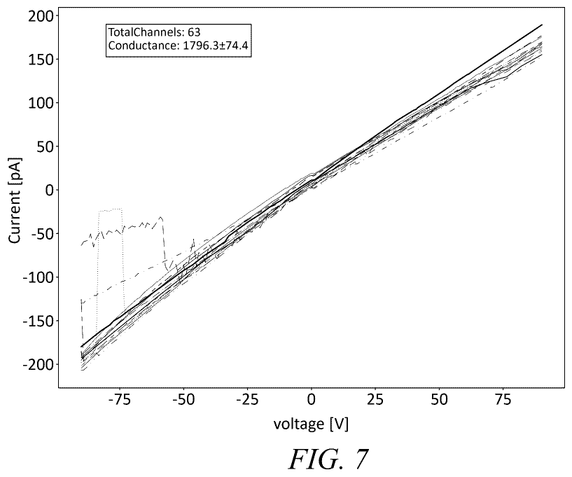

As an example, FIG. 7 is a current-voltage plot of a MspA pore protein. Based on the current-voltage relationship shown in FIG. 7, the conductivity of a MspA pore protein is around 1800 pS in IM KCL (see the slope of the majority of the traces). Gating behavior of the MspA pore protein can be observed in the negative voltage regime (see the outlier traces), in which the conductivity is reduced by a factor of 3 or more. FIG. 7 also shows that the gating voltage threshold is about −50 mV.

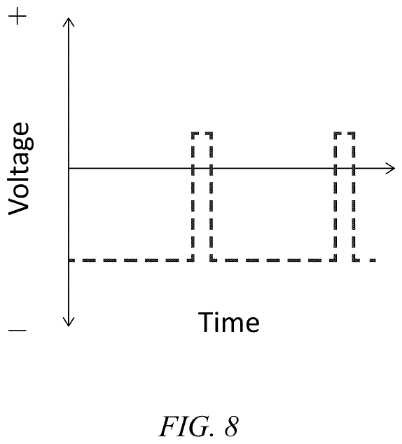

Using a bias voltage that has a small magnitude (more positive than the threshold) allows for only a slow recharging step in the whole sequencing workflow and is very time-consuming. The efficiency of the sequencing workflow in terms of throughput can be improved by finding faster ways of recharging. To make recharging efficient, positive voltage pulses may be applied during recharging to reset the pore back to the high conductivity state. Thus, in some embodiments, a pulsed voltage waveform that contains positive pulses in a negative constant voltage may be used to improve recharging efficiency. FIG. 8 shows such an example pulsed voltage waveform. In some embodiments, the negative constant portion within the pulsed waveform may have a voltage of about −2 mV, −5 mV, −10 mV, −20 mV, −50 mV, −100 mV, −200 mV, −500 mV, −1000 mV, −2000 mV, −5000 mV, or any value therebetween. In some embodiments, the period between pulses (i.e., the duration of the negative constant portion) within the pulsed waveform may have a duration of about 5 ms, 10 ms, 20 ms, 50 ms, 100 ms, 200 ms, 500 ms, 1000 ms, 2000 ms, 5000 ms, or any value therebetween. In some embodiments, the pulses within the pulsed waveform may have a voltage of about 1 mV, 2 mV, 5 mV, 10 mV, 20 mV, 50 mV, 100 mV, 200 mV, 500 mV, 1000 mV, 2000 mV, or any value therebetween. The pulses may have a duration of about 1 μs, 10 μs, 100 μs, 0.5 ms, 1 ms, 2 ms, 5 ms, 10 ms, 20 ms, 50 ms, 100 ms, 200 ms, 500 ms, 1000 ms, or any value therebetween.

In some embodiments, efficient recharging may be achieved by using a calibrated pulsed waveform. Typically, application of voltages more negative than the gating threshold (i.e., having a bigger magnitude) will cause the pore to gate, given a long enough recharge period of time. Applying positive voltage pulses within this recharge period may un-gate the pore for a short amount of time. By adjusting the durations, timings and amplitudes of the pulses, a voltage waveform more negative than the gating threshold can be applied without the pore spending a significant amount of time in a gated state. Thus, the net charge passed in the negative direction can be increased compared to, for example, applying a constant bias that is less negative than the gating threshold (i.e., having a smaller magnitude). As such, the time needed to recharge the wells may be decreased, improving the performance of the sequencing workflow and the data throughput.

The nanopore sequencing throughput is mainly affected by the recharging time needed. In the example sequencing process illustrated in FIG. 4, various states of operation are shown. In some cases, the “capturing state” occurs for about several seconds to minutes in 1 M KCL, with a conductivity range around 1800 pS, passing positive current through the pore. In some cases, the “sequencing state” occurs for about several minutes to hours (depending on the polynucleotide template length), with conductivities around 500 pS in 1 M KCL, passing positive current through the nanopore. The total charge passed during the sequencing state depends on the sequencing method. In some cases, the “eject state” takes about several seconds, passing negative current, with conductivities reduced due to gating by at least a factor of 3 and up to a factor of 10. In some cases, there may be another “stripping state” (not shown in FIG. 4) that occurs for about several seconds at a conductivity range around 500 pS in 1 M KCl, passing positive current. Therefore, the amount of time needed for the “recharging state” is mainly determined by how long the “capturing state” and the “sequencing state” take. In some cases, the recharging state may require about several minutes to hours and may occupy about 10% to 90% of the time needed for completing one sequencing cycle.

### EXAMPLES

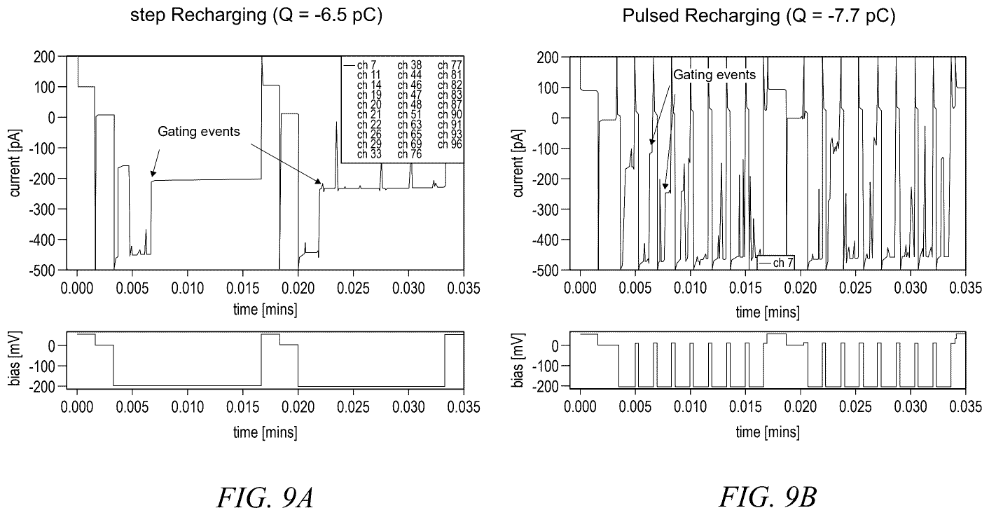

FIG. 9A and FIG. 9B show a direct comparison between the step recharging scenario versus the pulsed recharging scenario. The data shows that more net charge is passed with pulsed recharging in the same amount of time.

The step recharging scenario shown in FIG. 9A triggers gating and reduces the net passed negative charge to balance the overall charges during the sequencing workflow. The net passed negative charge in FIG. 9A is −6.5 pC.

While gating also occurs in the pulsed recharging scenario as shown in FIG. 9B, the positive pulses un-gates the pores quickly, and as a result the net passed negative charge is higher given the same amount of time. The net passed negative charge in FIG. 9A is −7.7 pC.

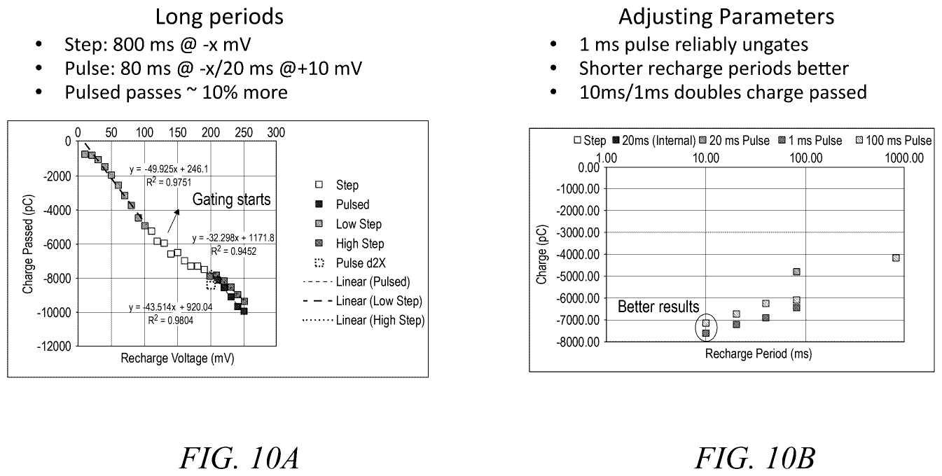

FIG. 10A and FIG. 10B show a summary of experimental data testing the pulsed recharging scenario versus the step recharging scenario.

FIG. 10A shows the net charge passed versus “recharge voltage”, which represents either the magnitude of the step in the step recharging scenario or the magnitude of the negative constant portion within a pulsed waveform in the pulsed recharging scenario. The step lasts for 800 ms. The period between pulses lasts for 80 ms. The pulses have a magnitude of 10 mV and a duration of 20 ms. The data shows that the pulsed recharging scenario passes at least about 10% more net negative charge through the nanopore than the step recharging scenario.

FIG. 10B shows the net charge passed with various pulse duration and period between pulses. The data shows that with shorter period between pulses, more charge gets passed. Moreover, 1 ms seems to be a better pulse duration in terms of allowing a higher amount of net of charge passed.

### ADDITIONAL EXAMPLES

Example 1. A method of controlling a nanopore in an electrolyte, the method comprising:
a. applying a first voltage across the nanopore; and
b. applying a second voltage across the nanopore, the second voltage having an opposite sign compared to the first voltage.

Example 2. The method as defined in Example 1, wherein applying a first voltage across the nanopore drives electrolyte ions through the nanopore.

Example 3. The method as defined in Example 1, wherein applying a second voltage across the nanopore results in conformational changes and/or changes in the conductance of the nanopore.

Example 4. The method as defined in Example 1, wherein step a and step b are repeated.

Example 5. The method as defined in Example 1, wherein no polynucleotide or moieties attached thereto are near or in the nanopore during application of the first voltage or the second voltage.

Example 6. The method as defined in Example 1, wherein a polynucleotide and/or moieties attached thereto are near or in the nanopore during application of the first voltage or the second voltage.

Example 7. The method as defined in Example 1, wherein the first voltage and the second voltage are applied in a controlled voltage mode.

Example 8. The method as defined in Example 1, wherein the first voltage and the second voltage are applied in a controlled current mode.

Example 9. A method of controlling a nanopore in an electrolyte, the method comprising:

applying a pulsed voltage waveform across the nanopore, wherein the maximum and minimum of the pulsed voltage waveform have opposite signs.

Example 10. The method as defined in Example 9, wherein applying a pulsed voltage waveform across the nanopore modulates gating of the nanopore.

Example 11. The method as defined in Example 9, wherein no polynucleotide or moieties attached thereto are near or in the nanopore during application of the pulsed voltage waveform.

Example 12. The method as defined in Example 9, wherein a polynucleotide and/or moieties attached thereto are near or in the nanopore during application of the pulsed voltage waveform.

Example 13. The method as defined in Example 9, wherein the pulsed voltage waveform is applied in a controlled voltage mode.

Example 14. The method as defined in Example 9, wherein the pulsed voltage waveform is applied in a controlled current mode.

Example 15. A method of controlling a nanopore unit cell, the nanopore unit cell comprising a nanopore in an electrolyte, the nanopore unit cell having a cis side and a trans side, the method comprising:
a. applying a negative voltage between the cis side and the trans side; and
b. applying a positive voltage between the cis side and the trans side.

Example 16. The method as defined in Example 15, wherein applying a negative voltage between the cis side and the trans side drives electrolyte ions through the nanopore.

Example 17. The method as defined in Example 15, wherein applying a positive voltage between the cis side and the trans side results in conformational changes and/or changes in the conductance of the nanopore.

Example 18. The method as defined in Example 15, wherein step a and step b are repeated.

Example 19. The method as defined in Example 15, wherein no polynucleotide or moieties attached thereto are near or in the nanopore during application of the negative voltage or the positive voltage.

Example 20. The method as defined in Example 15, wherein a polynucleotide and/or moieties attached thereto are near or in the nanopore during application of the negative voltage or the positive voltage.

Example 21. The method as defined in Example 15, wherein the negative voltage and the positive voltage are applied in a controlled voltage mode.

Example 22. The method as defined in Example 15, wherein the negative voltage and the positive voltage are applied in a controlled current mode.

Example 23. A method of controlling a nanopore unit cell, the nanopore unit cell comprising a cis side, a trans side, and a nanopore between the cis side and the trans side, the method comprising:
applying a pulsed voltage waveform between the cis side and the trans side, wherein the maximum and minimum of the pulsed voltage waveform have opposite signs.

Example 24. The method as defined in Example 23, wherein applying a pulsed voltage waveform between the cis side and the trans side modulates gating of the nanopore.

Example 25. The method as defined in Example 23, wherein no polynucleotide or moieties attached thereto are near or in the nanopore during application of the pulsed voltage waveform.

Example 26. The method as defined in Example 23, wherein a polynucleotide and/or moieties attached thereto are near or in the nanopore during application of the pulsed voltage waveform.

Example 27. The method as defined in Example 23, wherein the pulsed voltage waveform is applied in a controlled voltage mode.

Example 28. The method as defined in Example 23, wherein the pulsed voltage waveform is applied in a controlled current mode.

Example 29. A method of sequencing a polynucleotide, comprising:
measuring an electrical signal in a nanopore unit cell when a polynucleotide and/or moieties attached thereto are near or in the nanopore to determine one or more nucleobases in the polynucleotide; and
controlling the nanopore unit cell according to the method of any of Examples 1-28.

Example 30. A method of recharging a nanopore unit cell comprising a cis side, a trans side, and a nanopore disposed between the cis side and the trans side, the method comprising:
applying a pulsed voltage waveform across the nanopore, wherein the pulsed voltage waveform comprises a first voltage to drive ionic current from the trans side to the cis side and a second voltage that reverses the ionic current flow.

Example 31. The method as defined in Example 30, wherein applying a pulsed voltage waveform across the nanopore modulates gating of the nanopore.

Example 32. The method as defined in Example 30, wherein no polynucleotide or moieties attached thereto are near or in the nanopore during application of the pulsed voltage waveform.

Example 33. The method as defined in Example 30, wherein a polynucleotide and/or moieties attached thereto are near or in the nanopore during application of the pulsed voltage waveform.

Example 34. The method as defined in Example 30, wherein the pulsed voltage waveform is applied in a controlled voltage mode.

Example 35. The method as defined in Example 30, wherein the pulsed voltage waveform is applied in a controlled current mode.

Example 36. A method of sequencing a polynucleotide, comprising:
measuring an electrical signal in a nanopore unit cell when a polynucleotide and/or moieties attached thereto are near or in the nanopore to determine one or more nucleobases in the polynucleotide; and
recharging the nanopore unit cell according to the method of any of Examples 30-35.

### Additional Notes

It should be appreciated that all combinations of the foregoing embodiments and additional embodiments discussed in greater detail below (provided such embodiments are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular embodiments disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 nm to about 20 nm should be interpreted to include not only the explicitly recited limits of from about 2 nm to about 20 nm, but also to include individual values, such as about 3.5 nm, about 8 nm, about 18.2 nm, etc., and sub-ranges, such as from about 5 nm to about 10 nm, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an energy storage system described herein can be provided separately, or integrated together (e.g., packaged together, or attached together) to form an energy storage system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method of sequencing nucleic acids using an array of nanopore cells, each nanopore cell comprising a protein nanopore, a trans well and a trans electrode, the method comprising controlling at least one nanopore cell by:
(a) applying a sequencing waveform across the protein nanopore of the at least one nanopore cell, resulting in depletion of electrolytes in the trans well of the at least one nanopore cell; and
(b) applying a pulsed recharging waveform across the protein nanopore, resulting in replenishment of electrolytes in the trans well.

2. The method as defined in claim 1, wherein the sequencing waveform is DC, stepped, pulsed, AC, sinusoidal, or combinations thereof.

3. The method as defined in claim 1, wherein the sequencing waveform comprises a negative constant voltage.

4. The method as defined in claim 1, wherein the minimum of the sequencing waveform is more negative than the gating threshold voltage of the protein nanopore.

5. The method as defined in claim 1, wherein applying the pulsed recharging waveform across the protein nanopore results in conformational changes and/or changes in the conductance of the protein nanopore.

6. The method as defined in claim 1, wherein applying the pulsed recharging waveform across the protein nanopore modulates gating of the protein nanopore.

7. The method as defined in claim 1, wherein the sequencing waveform drives ionic current from the trans side to the cis side of the at least one nanopore cell and the pulsed recharging waveform reverses the ionic current flow.

8. The method as defined in claim 1, wherein the sequencing waveform and the pulsed recharging waveform have opposite signs.

9. The method as defined in claim 1, wherein the pulsed recharging waveform is a positive voltage pulse having a shorter duration than the sequencing waveform.

10. The method as defined in claim 1, wherein the pulsed recharging waveform has a rectangular shape.

11. The method as defined in claim 1, wherein step (a) and step (b) are repeated.

12. The method as defined in claim 1, wherein no polynucleotide or moieties attached thereto are near or in the nanopore during application of the pulsed recharging waveform.

13. The method as defined in claim 1, wherein a polynucleotide and/or moieties attached thereto are near or in the nanopore during application of the sequencing waveform.

14. The method as defined in claim 1, wherein the sequencing waveform and/or the pulsed recharging waveform are applied in a controlled voltage mode.

15. The method as defined in claim 1, wherein the sequencing waveform and/or the pulsed recharging waveform are applied in a controlled current mode.

16. The method as defined in claim 1, claims, further comprising measuring an electrical signal in the at least one nanopore cell when a polynucleotide and/or moieties attached thereto are near or in the protein nanopore of the at least one nanopore cell to determine one or more nucleobases in the polynucleotide.

17. The method as defined in claim 1, wherein the trans electrode of each nanopore cell is individually addressable.

18. The method as defined in claim 1, wherein each nanopore cell is independently controlled.

19. The method as defined in claim 1, wherein only one nanopore cell in the array is recharged at a given time.

20. The method as defined in claim 1, wherein multiple nanopore cells in the array are recharged simultaneously.
